(19) United States
(12) Patent Application Publication
(10) Pub. No.: US 2016/0193155 A1
(43) Pub. Date: Jul. 7, 2016

(54) PULSATILE-RELEASE DOSAGE FORM

(71) Applicant: SUN PHARMACEUTICAL INDUSTRIES LIMITED, Mumbai, Maharashtra (IN)

(72) Inventors: Vivek SHARMA, Ujjain (IN); Varinder KUMAR, Mohali (IN); Lalit Kumar KHURANA, Chandigarh (IN); Shavej AHMAD, Lucknow (IN); Romi Barat SINGH, Varanasi (IN); Ajay Kumar SINGLA, Gurgaon (IN)

(21) Appl. No.: 14/915,699

(22) PCT Filed: Aug. 28, 2014
(86) PCT No.: PCT/IB2014/064133

§ 371 Date: Mar. 1, 2016

(30) Foreign Application Priority Data
Sep. 2, 2013 (IN) 2582/DEL/2013

(51) Int. Cl. A61K 9/48; A61K 31/196; A61K 31/4178; A61K 9/28; A61K 9/20

(52) U.S. Cl. CPC A61K 9/4891; A61K 9/2866; A61K 9/2893; A61K 9/2095; A61K 31/4178; A61K 31/196

(57) ABSTRACT

The present invention relates to pulsatile-release dosage forms wherein said dosage forms provide pH-independent pulsatile-release of a drug after a predetermined lag time. It also relates to processes for the preparation of said pulsatile-release dosage forms.

### FIELD OF THE INVENTION

The present invention relates to pulsatile-release dosage forms wherein said dosage forms provide pH-independent pulsatile-release of a drug after a predetermined lag time. It also relates to processes for the preparation of said pulsatile-release dosage forms.

### BACKGROUND OF THE INVENTION

Several diseases show circadian rhythms in their pathophysiology. These include cardiovascular diseases such as hypertension and acute myocardial infarction; neurological diseases such as attention-deficit syndrome and Parkinson's syndrome; arthritis; asthma; cancer; peptic ulcer; diabetes mellitus; and hypercholesterolemia.

Pulsatile-release dosage forms are highly promising and advantageous over conventional immediate-release and extended-release dosage forms for the treatment of diseases that show a predictable cyclic rhythm, because the time of drug delivery plays a key role in effective treatment of these diseases. Pulsatile-release dosage forms deliver the drug at an appropriate time as per the pathophysiological need of the disease, leading to an improved therapeutic efficacy and better patient compliance. These dosage forms are designed according to the circadian rhythm of the body so that the drug is released as a pulse after a predetermined lag time.

U.S. Publication No. 2011/0189286 discloses a gastro-retentive pulsatile delivery system for the sustained delivery of valsartan, comprising an immediate-release (IR) component that provides a first pulse of valsartan and a modified-release (MR) component that provides additional pulse of valsartan, wherein valsartan is in a solubility enhanced form such that the pulsatile delivery of valsartan occurs in a therapeutically effective and gastro-retentive manner.

U.S. Pat. No. 6,663,888 discloses a histamine H₂ antagonist pharmaceutical dosage form providing a bi-modal pulsatile-release profile comprising: immediate-release (IR) beads comprising an active-containing core particle and timed pulsatile-release (TPR) beads. The TPR beads comprise an active-containing core particle and a pulse coating comprising a water-insoluble polymer and an enteric polymer surrounding said core. The IR beads provide a therapeutically effective amount of active to treat gastric acid secretions and TPR beads provide a delayed dose of active which provides a therapeutically effective amount of active to treat midnight GERD.

U.S. Pat. No. 7,413,751 discloses a gastric retained dosage form comprising losartan and a hydrophilic polymer such as polyethylene oxide, hydroxyethylcellulose, and hydroxypropylmethylcellulose. Losartan is administered from this dosage form for a period of at least 5 hours and at least 40% w/w of the losartan is retained in the dosage form after 1 hour.

U.S. Publication No. 2005/0276853 discloses a chronotherapeutic pharmaceutical formulation comprising a core of an active ingredient and a delayed-release compression coating of a natural or synthetic gum applied onto the surface of the core.

There remains a need for a pulsatile-release dosage form in which the release of a drug is delayed for as many hours as is desired, based on the pathophysiology of the disease. The present inventors have now developed a novel pulsatile-release dosage form that provides release of the drug after a predetermined lag time. The dosage form of the present invention is therefore beneficial, as it avoids the unnecessary exposure of the target organ to the drug during those times when therapeutic effect of the drug is not required. For instance, for the treatment of certain disease conditions, said pulsatile-release dosage form may be administered prior to sleep so that the drug is released well after the time of administration, for example, during morning hours. Such administration results in effective treatment of early morning pathological symptoms in certain disease conditions like hypertension and arthritis. Additionally, the pulsatile-release dosage form of the present invention can be designed so as to target the drug-release at a particular site, such as the lower portion of the gastro-intestinal tract.

Further, the release of the drug from the pulsatile-release dosage form of the present invention is independent of the pH of the gastro-intestinal tract. The pulsatile-release dosage form of the present invention is relatively simple, easy to manufacture, and is functionally reproducible.

### SUMMARY OF THE INVENTION

The present invention relates to pulsatile-release dosage forms which provide pH independent-release of a drug after a predetermined lag time. The pulsatile-release dosage forms comprise: (i) a core comprising a drug and one or more pharmaceutically acceptable excipients; and (ii) a coating layer over said core comprising a water-soluble polymer, a water-insoluble polymer, a small molecular weight water-insoluble excipient, and one or more pharmaceutically acceptable coating additives. Further, the present invention also includes processes for the preparation of said pulsatile-release dosage forms.

### DETAILED DESCRIPTION OF THE INVENTION

A first aspect of the present invention provides a pulsatile-release dosage form comprising:
(a) a core comprising a drug and one or more pharmaceutically acceptable excipients; and
(b) a coating layer over said core comprising a water-soluble polymer, a water-insoluble polymer, a small molecular weight water-insoluble excipient, and one or more pharmaceutically acceptable coating additives.

According to one embodiment of this aspect, there is provided a pulsatile-release dosage form comprising:
(a) a core comprising a drug and one or more pharmaceutically acceptable excipients; and
(b) a coating layer over said core comprising hydroxypropyl methyl cellulose, ethyl cellulose, magnesium stearate, and one or more pharmaceutically acceptable coating additives.

According to another embodiment of this aspect, there is provided a pulsatile-release dosage form comprising:
(a) a core comprising a drug and one or more pharmaceutically acceptable excipients; and
(b) a coating layer over said core comprising polyvinylpyrrolidone, ethyl cellulose, magnesium stearate, and one or more pharmaceutically acceptable coating additives.

A second aspect of the present invention provides a pulsatile-release dosage form comprising:
(a) a core comprising a drug and one or more pharmaceutically acceptable excipients; and
(b) a coating layer over said core comprising a water-soluble polymer, a water-insoluble polymer, a small molecular weight water-insoluble excipient, and one or more pharmaceutically acceptable coating additives,
   wherein the core further comprises one or more rate-controlling polymers.

According to one embodiment of this aspect, there is provided a pulsatile-release dosage form comprising:
(a) a core comprising a drug and one or more pharmaceutically acceptable excipients; and
(b) a coating layer over said core comprising hydroxypropyl methyl cellulose, ethyl cellulose, magnesium stearate, and one or more pharmaceutically acceptable coating additives,
   wherein the core further comprises hydroxypropyl methyl cellulose.

A third aspect of the present invention provides a pulsatile-release dosage form comprising:
(a) a core comprising a drug and one or more pharmaceutically acceptable excipients; and
(b) a coating layer over said core comprising a water-soluble polymer, a water-insoluble polymer, a small molecular weight water-insoluble excipient, and one or more pharmaceutically acceptable coating additives,
   wherein the core further comprises one or more gastro-retentive polymers.

According to one embodiment of this aspect, there is provided a pulsatile-release dosage form comprising:
(a) a core comprising a drug and one or more pharmaceutically acceptable excipients; and
(b) a coating layer over said core comprising hydroxypropyl methyl cellulose, ethyl cellulose, magnesium stearate, and one or more pharmaceutically acceptable coating additives,
   wherein the core further comprises Kollicoat® Protect and Carbopol®.

A fourth aspect of the present invention provides a process for the preparation of a pulsatile-release dosage form, wherein the process comprises the steps of:
(a) blending/granulating a drug and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the tablet core of step (b) to form the pulsatile-release dosage form.

A fifth aspect of the present invention provides a process for the preparation of a pulsatile-release dosage form, wherein the process comprises the steps of:
(a) blending/granulating a drug, one or more rate-controlling polymers, and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the tablet core of step (b) to form the pulsatile-release dosage form.

A sixth aspect of the present invention provides a process for the preparation of a pulsatile-release dosage form, wherein the process comprises the steps of:
(a) blending/granulating a drug, one or more gastro-retentive polymers, and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the tablet core of step (b) to form the pulsatile-release dosage form.

A seventh aspect of the present invention provides a process for the preparation of a pulsatile-release dosage form, wherein the process comprises the steps of:
(a) blending/granulating a drug and one or more pharmaceutically acceptable excipients;
(b) filling the blends/granules of step (a) into a suitable sized capsule to form a capsule core;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the capsule core of step (b) to form the pulsatile-release dosage form.

An eighth aspect of the present invention provides a process for the preparation of a pulsatile-release dosage form, wherein the process comprises the steps of:
(a) blending/granulating a drug, one or more rate-controlling polymers, and one or more pharmaceutically acceptable excipients;
(b) filling the blends/granules of step (a) into a suitable sized capsule to form a capsule core;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the capsule core of step (b) to form the pulsatile-release dosage form.

A ninth aspect of the present invention provides a process for the preparation of a pulsatile-release dosage form, wherein the process comprises the steps of:
(a) blending/granulating a drug, one or more gastro-retentive polymers, and one or more pharmaceutically acceptable excipients;
(b) filling the blends/granules of step (a) into a suitable sized capsule to form a capsule core;
(c) dissolving/dispersing a water-soluble polymer, a water-insoluble polymer, a small molecular weight water-insoluble excipient, and one or more pharmaceutically acceptable coating additives in a suitable solvent; and
(d) applying the coating composition of step (c) over the capsule core of step (b) to form the pulsatile-release dosage form.

The term “pulsatile-release dosage form”, as used herein, refers to a dosage form designed in such a way so that either some amount of the drug or the entire amount of the drug is released after a predetermined period of time, i.e., lag time, upon administration of the dosage form.

The term “core”, as used herein, refers to tablets, capsules, granules, caplets, spheroids, and pellets, preferably tablets and capsules.

The coating layer delays the release of the drug from the core for a predetermined period of time and irrespective of the pH of the medium to which it is exposed. It has been surprisingly observed that incorporation of a small molecular weight water-insoluble excipient into the coating blend of a water-soluble polymer and a water-insoluble polymer affords the desired lag time. The small molecular weight water-insoluble excipient does not allow the water-insoluble polymer to form a continuous film and the water-soluble polymer to form continuous pore channels. As a result, when such a dosage form comes in contact with the fluids of the gastro-intestinal tract, the water-soluble polymer gets solubilized. The channels formed by the water-soluble polymer are however, not continuous, hence, drug release does not occur. Also, as the water-insoluble polymer does not form a continuous film, the film strength is low and it disintegrates after a predetermined lag time, after which some of or the entire amount of the drug gets released. Further, the release of drug from said pulsatile-release dosage form is independent of the pH of the gastro-intestinal tract.

Further, when one or more rate-controlling polymers are incorporated in the core of said pulsatile-release dosage form, some amount of drug will be released as a pulse after a predetermined lag time and the remaining amount of the drug will be released continuously over a prolonged period of time, thereby rendering extended-release benefits.

Alternatively, when one or more gastro-retentive polymers are incorporated in the core of said pulsatile-release dosage form, the dosage form would be retained in the upper gastro-intestinal tract for a prolonged period of time. Subsequently, after a predetermined lag time, the drug is released in the upper gastro-intestinal tract. Such a pulsatile-release dosage form is suitable for drugs that are preferentially absorbed from the upper part of the gastro-intestinal tract.

The lag time provided by said pulsatile-release dosage form can be controlled by the amount and type of water-soluble polymer, water-insoluble polymer, and small molecular weight water-insoluble excipient employed in the coating layer, as well as the thickness of the coating layer.

Examples of drugs which may be included in the pulsatile-release dosage form of the present invention include, but are not limited to, anti-hypertensive drugs like losartan, olmesartan, telmisartan, valsartan, clonidine, guanabenz, methyldopa, moxonidine, bumetanide, ethacrynic acid, furosemide, torsemide, epitizide, hydrochlorothiazide, chlorothiazide, bendroflumethiazide, indapamide, chlorthalidone, metolazone, amiloride, triamterene, spironolactone, atenolol, metoprolol, nadolol, nebivolol, oxprenolol, pindolol, propranolol, timolol, doxazosin, phentolamine, indoramin, phenoxybenzamine, prazosin, terazosin, tolazoline, bucindolol, carvedilol, labetalol, amlodipine, cilnidipine, felodipine, isradipine, lercanidipine, nicardipine, nifedipine, nimodipine, nitrendipine, diltiazem, verapamil, captopril, enalapril, fosinopril, lisinopril, perindopril, quinapril, ramipril, trandolapril, benazepril, candesartan, eprosartan, and irbesartan; anti-inflammatory drugs like diclofenac, aspirin, celecoxib, diflunisal, etodolac, fenoprofen, flurbiprofen, ibuprofen, indomethacin, ketoprofen, ketorolac, meloxicam, nabumetone, naproxen, oxaprozin, piroxicam, salsalate, sulindac, and tolmetin; anti-hyperlipidemic drugs like simvastatin, atorvastatin, cerivastatin, fluvastatin, lovastatin, mevastatin, pitavastatin, pravastatin, rosuvastatin, and niacin; anti-asthmatic drugs like theophylline, tulobuterol, salbutamol, femoterol, terbutalin, ipratropium bromide, oxitropium, aminophylline, beclomethasone, betamethasone, prednisolone, sodium chromoglycate, nedocromil sodium, zafirleukast, and zileuton; glucocorticoids like hydrocortisone, cortisone, prednisone, prednisolone, methylprednisolone, dexamethasone, betamethasone, triamcinolone, fludrocortisone acetate, and aldosterone; anti-ulcer drugs like omeprazole, famotidine, ranitidine, cimetidine, pantoprazole, esomeprazole, lansoprazole, and rabeprazole; anti-tumor drugs like oxaliplatin, fluorouracil, leucovorin, irinotecan, cisplastin, doxorubicin, methotrexate, folinic acid, mercaptopurine, docetaxel, and paclitaxel; anti-Parkinson's drugs like carbidopa, levodopa, and entacapone; or combinations thereof. The dose of any drug may depend upon the individual drug used in the pulsatile-release dosage form of the present invention.

Suitable water-soluble polymers are selected from the group consisting of hydroxypropyl methyl cellulose, hydroxyethyl cellulose, hydroxypropyl cellulose, carboxymethyl cellulose, polyethylene glycol, polyethylene oxide, polysaccharides, polyvinylpyrrolidone, polyvinyl alcohol, and combinations thereof.

Suitable water-insoluble polymers are selected from the group consisting of cellulose ethers such as ethyl cellulose; cellulose esters such as cellulose acetate; polyvinyl acetate; and combinations thereof.

Suitable small molecular weight water-insoluble excipients are selected from the group consisting of magnesium stearate, calcium stearate, cetyl alcohol, glyceryl behenate, talc, stearic acid, glyceryl monostearate, hydrogenated vegetable oil, hydrogenated castor oil, magnesium aluminum silicate, aluminum stearate, and combinations thereof.

The term “rate-controlling polymer”, as used herein, refers to the agent that helps to control the release of drug from the core. The rate-controlling polymer is selected from the group consisting of cellulose derivatives such as hydroxypropyl methylcellulose available in various grades (e.g., Methocel® K15 MCR and Methocel® E5), hydroxypropyl cellulose, carboxymethyl cellulose, and ethyl cellulose; polysaccharides such as alginate, xanthan gum, carrageenan, and starch; acrylic acid polymers like carbomer, polymethacrylate, poly(hydroxy ethyl methacrylate), poly(methyl methacrylate), and poly(hydroxy ethyl methacrylate-co methyl methacrylate); polyethylene oxides and co-polymers thereof; polypropylene and co-polymers thereof; and combinations thereof.

The term “gastro-retentive polymer”, as used herein, refers to the agent that helps to retain the dosage form in the stomach for a prolonged period of time. The gastro-retentive polymer is selected from the group consisting of polyvinyl alcohol-polyethylene glycol graft copolymers available under the trade names Kollicoat® Protect and Kollicoat® IR; acrylic acid polymers available under the trade name Carbopol®; and combinations thereof.

The core of the present invention comprises one or more pharmaceutically acceptable excipients that are routinely used in pharmaceutical dosage forms and are selected from the group consisting of fillers, binders, disintegrants, lubricants/glidants, and combinations thereof.

Suitable fillers are selected from the group consisting of microcrystalline cellulose, silicified microcrystalline cellulose, lactose, corn starch, glucose, calcium carbonate, calcium phosphate dibasic, calcium phosphate tribasic, calcium sulphate, starch, pregelatinized starch, lactitol, mannitol, sorbitol, and combinations thereof.

Suitable binders are selected from the group consisting of methyl cellulose, ethyl cellulose, hydroxyethylcellulose, hydroxypropyl cellulose, hydroxypropyl methylcellulose, carboxymethyl cellulose sodium, acacia, guar gum, alginic acid, acrylic acid polymer, dextrin, maltodextrin, magnesium aluminum silicate, polymethacrylates, polyvinylpyrrolidone, gelatin, starch, and combinations thereof.

Suitable disintegrants are selected from the group consisting of hydroxypropyl cellulose, microcrystalline cellulose, croscarmellose sodium, crospovidone, magnesium aluminum silicate, methylcellulose, sodium alginate, sodium starch glycolate, starch, mannitol, alginic acid, and combinations thereof.

Suitable lubricants/glidants are selected from the group consisting of colloidal silicon dioxide, stearic acid, magnesium stearate, zinc stearate, calcium stearate, talc, hydrogenated castor oil, vegetable oil, polyethylene glycol, polyvinyl alcohol, sodium benzoate, sodium stearylfumarate, magnesium oxide, poloxamer, sodium lauryl sulphate, polyoxyethylene monostearate, cocoa butter, mineral oil, polysaccharides, and combinations thereof.

The coating layer of the present invention comprises one or more pharmaceutically acceptable coating additives routinely used in the pharmaceutical dosage forms selected from the group consisting of plasticizers, opacifiers, anti-tacking agents, coloring agents, and combinations thereof.

Suitable plasticizers are selected from the group consisting of triethyl citrate, dibutyl sebacate, triacetin, acetylated triacetin, acetyl tributyl citrate, acetyl triethyl citrate, glycerin, sorbitol, diethyl oxalate, diethyl phthalate, diethyl malate, diethyl fumarate, dibutyl succinate, diethyl malonate, dioctyl phthalate, tributyl citrate, glyceryl tributyrate, diacetylated monoglyceride, rapeseed oil, olive oil, sesame oil, and combinations thereof.

Suitable opacifiers are selected from the group consisting of titanium dioxide, silicon dioxide, manganese dioxide, iron oxide, and combinations thereof.

Suitable anti-tacking agents are selected from the group consisting of talc, magnesium stearate, calcium stearate, stearic acid, silica, glyceryl monostearate, and combinations thereof.

Suitable coloring agents are selected from the group consisting of FD&C (Federal Food, Drug and Cosmetic Act) approved coloring agents; natural coloring agents; natural juice concentrates; pigments such as titanium dioxide, iron oxide, and zinc oxide; and combinations thereof.

Examples of solvents used for granulation or coating include ethanol, water, methylene chloride, isopropyl alcohol, acetone, methanol, or combinations thereof.

The core can be prepared by usual blending or granulating techniques and the resultant blends/granules can be compressed into a tablet using a conventional tabletting process, or filled into a capsule of a suitable size using any conventional process.

Coating may be performed by applying the coating composition as a solution, suspension, or blend using any conventional coating technique known in the art such as spray coating in a conventional coating pan, a fluidized bed processor, dip coating, or compression coating. The percentage of the coating build-up may be varied depending upon the desired lag time.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Examples 1 and 2

Quantity (mg/tablet)

| | | |
|---|---|---|
| Ingredients | Example 1 | Example 2 |

Core

| | | |
|---|---|---|
| Losartan potassium | 100.00 | 100.00 |
| Microcrystalline cellulose | 150.00 | 150.00 |
| Lactose | 120.00 | 120.00 |
| Hydroxypropyl methyl cellulose | 70.00 | 70.00 |
| Crospovidone | 50.00 | 50.00 |
| Colloidal silicon dioxide | 5.00 | 5.00 |
| Magnesium stearate | 5.00 | 5.00 |
| Core weight | 500.00 | 500.00 |

Coating Layer

| | | |
|---|---|---|
| Ethyl cellulose | 10.56 | 31.68 |
| Hydroxypropyl methyl cellulose | 3.96 | — |
| Polyvinylpyrrolidone | — | 2.63 |
| Triethyl citrate | 5.28 | — |
| Triacetin | — | 0.67 |
| Magnesium stearate | 46.20 | 31.02 |
| Talc | 6.00 | 6.00 |
| Titanium dioxide | 3.00 | 3.00 |
| Ethanol | q.s. | q.s. |
| Purified water | q.s. | q.s. |
| Coating Weight | 75.00 | 75.00 |
| Total Weight | 575.00 | 575.00 |

### Procedure:

### Core:

1. Losartan potassium, microcrystalline cellulose, lactose, hydroxypropyl methyl cellulose, crospovidone, and colloidal silicon dioxide were sifted through a suitable mesh and blended.
2. Magnesium stearate was mixed with the blend of step 1.
3. The resultant blend of step 2 was compressed into tablets using appropriate tooling.

### Coating Layer:

4. Ethyl cellulose, hydroxypropyl methyl cellulose/polyvinylpyrrolidone, and triethyl citrate/triacetin were dissolved in an ethanol and purified water mixture.
5. Magnesium stearate, talc, and titanium dioxide were dispersed into the solution of step 4.
6. The tablets of step 3 were coated in a pan coating machine using the coating solution of step 5 to form pulsatile-release dosage forms.

### In-Vitro Studies:

The tablets prepared according to Example 1 and Example 2 were subjected to dissolution studies in 900 mL of 0.1N HCl, pH 4.5 acetate buffer, and pH 6.8 phosphate buffer using a USP type II apparatus with a paddle speed at 50 rpm. The results of the release studies are represented in Table 1 and Table 2 below.

TABLE 1
Percentage (%) of In-Vitro Drug Release in USP Type II Apparatus
(Media: 900 mL of 0.1N HCl, pH 4.5 acetate buffer, and
pH 6.8 phosphate buffer at 50 rpm) From Tablets Prepared
According to Example 1

| Time | Drug Dissolved (Percent w/w) | | |
|---|---|---|---|
| (Hours) | 0.1N HCl | pH 4.5 Acetate Buffer | pH 6.8 Phosphate Buffer |
| 0.5 | 0.0 | 0.0 | 0.0 |
| 1.0 | 0.0 | 0.0 | 0.0 |
| 1.5 | 0.2 | 11.7 | 1.8 |
| 2.5 | 13.0 | 16.5 | 16.5 |
| 3.0 | 28.8 | 32.0 | 36.2 |
| 3.5 | 79.6 | 67.5 | 79.4 |
| 4.0 | 103.5 | 80.1 | 100.4 |
| 5.0 | — | 102.9 | — |

TABLE 2
Percentage (%) of In-Vitro Drug Release in USP Type II
Apparatus (Media: 900 mL of 0.1N HCl, pH 4.5 acetate buffer,
and pH 6.8 phosphate buffer at 50 rpm) From Tablets
Prepared According to Example 2

| Time | Drug Dissolved (Percent w/w) | | |
|---|---|---|---|
| (Hours) | 0.1N HCl | pH 4.5 Acetate Buffer | pH 6.8 Phosphate Buffer |
| 8 | 0.0 | 0.0 | 0.0 |
| 10 | 0.0 | 0.0 | 0.0 |
| 11 | 2.5 | 13.9 | 30.0 |
| 12 | 18.0 | 36.2 | 46.2 |
| 13 | 34.4 | 56.4 | 87.9 |
| 14 | 52.0 | 72.0 | 79.5 |
| 15 | 70.7 | 80.1 | 102.7 |
| 16 | 101.4 | 103.5 | — |

### Examples 3-6

| | Quantity (mg/tablet) | | | |
|---|---|---|---|---|
| Ingredients | Example 3 | Example 4 | Example 5 | Example 6 |

Core

| | | | | |
|---|---|---|---|---|
| Losartan potassium | 100.00 | 100.00 | 100.00 | 100.00 |
| Microcrystalline | 50.00 | 150.00 | 150.00 | 150.00 |
| cellulose | | | | |
| Lactose | 42.00 | 120.00 | 120.00 | 120.00 |
| Hydroxypropyl methyl | 42.00 | 70.00 | 70.00 | 70.00 |
| cellulose | | | | |
| Crospovidone | 60.00 | 50.00 | 50.00 | 50.00 |
| Colloidal silicon dioxide | 3.00 | 5.00 | 5.00 | 5.00 |
| Magnesium stearate | 3.00 | 5.00 | 5.00 | 5.00 |
| Core Weight | 300.00 | 500.00 | 500.00 | 500.00 |

Coating Layer

| | | | | |
|---|---|---|---|---|
| Ethylcellulose | 12.63 | 8.00 | 16.00 | 24.00 |
| Hydroxypropyl methyl | 3.16 | 4.00 | 8.00 | 12.00 |
| cellulose | | | | |
| Dibutyl sebacate | 0.95 | — | — | — |
| Triethyl citrate | — | 1.00 | 2.00 | 3.00 |
| Magnesium stearate | 9.47 | 9.00 | 18.00 | 27.00 |
| Talc | 2.37 | 2.00 | 4.00 | 6.00 |
| Titanium dioxide | 1.42 | 0.88 | 1.75 | 2.63 |
| Iron oxide red | — | 0.12 | 0.25 | 0.38 |
| Ethanol | q.s. | q.s. | q.s. | q.s. |
| Purified water | q.s. | q.s. | q.s. | q.s. |
| Coating Weight | 30.00 | 25.00 | 50.00 | 75.01 |
| Total Weight | 330.00 | 525.00 | 550.00 | 575.01 |

### Procedure:

### Core:

1. Losartan potassium, microcrystalline cellulose, lactose, hydroxypropyl methyl cellulose, crospovidone, and colloidal silicon dioxide were sifted through a suitable mesh and blended.
2. Magnesium stearate was mixed with the blend of step 1.
3. The resultant blend of step 2 was compressed into tablets using appropriate tooling.

### Coating Layer:

4. Ethyl cellulose, hydroxypropyl methyl cellulose and dibutyl sebacate/triethyl citrate were dissolved in an ethanol and purified water mixture.
5. Magnesium stearate, talc, titanium dioxide, and iron oxide red (Examples 4, 5 and 6) were dispersed into the solution of step 4.
6. The tablets of step 3 were coated in a pan coating machine using the coating solution of step 5 to form pulsatile-release dosage forms.

### Example 7

| | |
|---|---|
| Ingredients | Quantity (mg/tablet) |

Core

| | |
|---|---|
| Diclofenac sodium | 100.00 |
| Microcrystalline cellulose | 80.00 |
| Lactose | 49.00 |
| Hydroxypropyl methyl cellulose | 15.00 |
| Crospovidone | 50.00 |
| Colloidal silicon dioxide | 3.00 |
| Magnesium stearate | 3.00 |
| Core Weight | 300.00 |

Coating Layer

| | |
|---|---|
| Ethyl cellulose | 25.266 |
| Hydroxypropyl methyl cellulose | 6.318 |
| Dibutyl sebacate | 1.896 |
| Magnesium stearate | 18.948 |
| Talc | 4.734 |
| Titanium dioxide | 2.838 |
| Ethanol | q.s. |
| Purified water | q.s. |
| Coating Weight | 60.000 |
| Total weight | 360.000 |

### Procedure:

### Core:

1. Diclofenac sodium, microcrystalline cellulose, lactose, hydroxyl propyl methyl cellulose, crospovidone, and colloidal silicon dioxide were sifted through a suitable mesh and blended.
2. Magnesium stearate was mixed with the blend of step 1.
3. The resultant blend of step 2 was compressed into tablets using appropriate tooling.

### Coating Layer:

4. Ethyl cellulose, hydroxypropyl methyl cellulose, and dibutyl sebacate were dissolved in an ethanol and purified water mixture.
5. Magnesium stearate, talc, and titanium dioxide were dispersed into the solution of step 4.
6. The tablets of step 3 were coated in a pan coating machine using the coating solution of step 5 to form pulsatile-release dosage forms.

### Example 8 and Example 9

Quantity (mg/tablet)

| | | |
|---|---|---|
| Ingredients | Example 8 | Example 9 |

Core

| | | |
|---|---|---|
| Losartan potassium | 100.00 | 100.00 |
| Microcrystalline cellulose | 50.00 | 50.00 |
| Methocel ® K15 MCR | 80.00 | 80.00 |
| Methocel ® E5 | 42.00 | 42.00 |
| Lactose | 22.00 | 22.00 |
| Colloidal silicon dioxide | 3.00 | 3.00 |
| Magnesium stearate | 3.00 | 3.00 |
| Core Weight | 300.00 | 300.00 |

Coating layer

| | | |
|---|---|---|
| Ethyl cellulose | 12.63 | 18.95 |
| Hydroxypropyl methyl cellulose | 3.16 | 4.74 |
| Dibutyl sebacate | 0.95 | 1.43 |
| Magnesium stearate | 9.47 | 14.20 |
| Talc | 2.37 | 3.55 |
| Titanium dioxide | 1.42 | 2.13 |
| Ethanol | q.s. | q.s. |
| Purified water | q.s. | q.s. |
| Coating Weight | 30.00 | 45.00 |
| Total Weight | 330.00 | 345.00 |

### Procedure:

### Core:

1. Losartan potassium, microcrystalline cellulose, lactose, Methocel® K15 MCR, Methocel® E5, and colloidal silicon dioxide are sifted through a suitable mesh and blended.
2. Magnesium stearate is mixed with the blend of step 1.
3. The resultant blend of step 2 is compressed into tablets using appropriate tooling.

### Coating Layer:

4. Ethyl cellulose, hydroxypropyl methyl cellulose, and dibutyl sebacate are dissolved in an ethanol and purified water mixture.
5. Magnesium stearate, talc, and titanium dioxide are dispersed into the solution of step 4.
6. The tablets of step 3 are coated in a pan coating machine using the coating solution of step 5 to form pulsatile-release dosage forms.

### Examples 10 and 11

Quantity (mg/tablet)

| | | |
|---|---|---|
| Ingredients | Example 10 | Example 11 |

Core

| | | |
|---|---|---|
| Diclofenac sodium | 50.00 | 50.00 |
| Microcrystalline cellulose | 15.00 | 15.00 |
| Methocel ® K15 MCR | 57.00 | 25.00 |
| Lactose | 25.00 | 57.00 |
| Colloidal silicon dioxide | 1.50 | 1.50 |
| Magnesium stearate | 1.50 | 1.50 |
| Core Weight | 150.00 | 150.00 |

Coating Layer

| | | |
|---|---|---|
| Ethyl cellulose | 6.315 | 12.63 |
| Hydroxypropyl methyl cellulose | 1.580 | 3.16 |
| Dibutyl sebacate | 0.475 | 0.95 |
| Magnesium stearate | 4.735 | 9.47 |
| Talc | 1.185 | 2.37 |
| Titanium dioxide | 0.710 | 1.42 |
| Ethanol | q.s. | q.s. |
| Purified water | q.s. | q.s. |
| Coating Weight | 15.000 | 30.00 |
| Total Weight | 165.00 | 180.00 |

### Procedure:

### Core:

1. Diclofenac sodium, microcrystalline cellulose, lactose, Methocel® K15MCR, and colloidal silicon dioxide are sifted through a suitable mesh and blended.
2. Magnesium stearate is mixed with the blend of step 1.
3. The resultant blend of step 2 is compressed into tablets using appropriate tooling.

### Coating Layer:

4. Ethyl cellulose, hydroxypropyl methyl cellulose, and dibutyl sebacate are dissolved in an ethanol and purified water mixture.
5. Magnesium stearate, talc, and titanium dioxide are dispersed into the solution of step 4.
6. The tablets of step 3 are coated in a pan coating machine using the coating solution of step 5 to form pulsatile-release dosage forms.

### Example 12

| | |
|---|---|
| | Quantity (mg/tablet) |
| Ingredients | Example 12 |

Core

| | |
|---|---|
| Losartan potassium | 100.00 |
| Mannitol | 300.00 |
| Kollicoat ® Protect | 340.00 |
| Carbopol ® | 250.00 |
| Colloidal silicon dioxide | 5.00 |
| Magnesium stearate | 5.00 |
| Core Weight | 1000.00 |

Coating Layer

| | |
|---|---|
| Ethyl cellulose | 32.00 |
| Hydroxypropyl methyl cellulose | 16.00 |
| Triethyl citrate | 4.00 |
| Magnesium stearate | 36.00 |
| Talc | 8.00 |
| Titanium dioxide | 3.50 |
| Iron oxide red | 0.50 |
| Ethanol | q.s. |
| Purified water | q.s. |
| Coating Weight | 100.00 |
| Total Weight | 1100.00 |

### Procedure:

### Core:

1. Losartan potassium, mannitol, Kollicoat® Protect, Carbopol®, and colloidal silicon dioxide are sifted through a suitable mesh and blended.
2. Magnesium stearate is mixed with the blend of step 1.
3. The resultant blend of step 2 is compressed into tablets using appropriate tooling.

### Coating Layer:

4. Ethyl cellulose, hydroxypropyl methyl cellulose, and triethyl citrate are dissolved in an ethanol and purified water mixture.
5. Magnesium stearate, talc, titanium dioxide, and iron oxide red are dispersed into the solution of step 4.
6. The tablets of step 3 are coated in a pan coating machine using the coating solution of step 5 to form pulsatile-release dosage forms.

### Example 13

| | |
|---|---|
| | Quantity (mg/capsule) |
| Ingredients | Example 13 |

Core

| | |
|---|---|
| Losartan potassium | 100.00 |
| Mannitol | 175.00 |
| Colloidal silicon dioxide | 5.00 |
| Core Weight | 280.00 |
| Gelatin band | q.s. |
| Weight of Filled Capsule | 400.00 |

Coating Layer

| | |
|---|---|
| Ethyl cellulose | 12.80 |
| Hydroxypropyl methyl cellulose | 6.40 |
| Triethyl citrate | 1.60 |
| Magnesium stearate | 14.40 |
| Talc | 3.20 |
| Titanium dioxide | 1.40 |
| Iron oxide red | 0.20 |
| Ethanol | q.s. |
| Purified water | q.s. |
| Coating Weight | 40.00 |
| Total Weight | 440.00 |

### Procedure:

### Core:

1. Losartan potassium, mannitol, and colloidal silicon dioxide are sifted through a suitable mesh and blended.
2. A suitable sized capsule is filled with the blend of step 1.
3. The resultant capsule is band-sealed with a gelatin solution.

### Coating Layer:

4. Ethyl cellulose, hydroxypropyl methyl cellulose, and triethyl citrate are dissolved in an ethanol and purified water mixture.
5. Magnesium stearate, talc, titanium dioxide, and iron oxide red are dispersed into the solution of step 4.
6. The capsules of step 3 are coated in a pan coating machine using the coating solution of step 5 to form pulsatile-release dosage forms.

We claim:

1. A pulsatile-release dosage form comprising:
(a) a core comprising a drug and one or more pharmaceutically acceptable excipients; and
(b) a coating layer over said core comprising a water-soluble polymer, a water-insoluble polymer, a small molecular weight water-insoluble excipient, and one or more pharmaceutically acceptable coating additives.

2. The pulsatile-release dosage form of claim 1, wherein the water-soluble polymer is selected from the group consisting of hydroxypropyl methyl cellulose, hydroxyethyl cellulose, hydroxypropyl cellulose, carboxymethyl cellulose, polyethylene glycol, polyethylene oxide, polysaccharides, polyvinylpyrrolidone, polyvinyl alcohol, and combinations thereof.

3. The pulsatile-release dosage form of claim 1, wherein the water-insoluble polymer is selected from the group consisting of ethyl cellulose, cellulose acetate, polyvinyl acetate, and combinations thereof.

4. The pulsatile-release dosage form of claim 1, wherein the small molecular weight water-insoluble excipient is selected from the group consisting of magnesium stearate, calcium stearate, cetyl alcohol, glyceryl behenate, talc, stearic acid, glyceryl monostearate, hydrogenated vegetable oil, hydrogenated castor oil, magnesium aluminum silicate, aluminum stearate, and combinations thereof.

5. The pulsatile-release dosage form of claim 1, wherein the coating layer comprises hydroxypropyl methyl cellulose, ethyl cellulose, magnesium stearate, and one or more pharmaceutically acceptable coating additives.

6. The pulsatile-release dosage form of claim 1, wherein the coating layer comprises polyvinylpyrrolidone, ethyl cellulose, magnesium stearate, and one or more pharmaceutically acceptable coating additives.

7. The pulsatile-release dosage form of claim 1, wherein the pharmaceutically acceptable excipients are selected from the group consisting of fillers, binders, disintegrants, lubricants/glidants, and combinations thereof.

8. The pulsatile-release dosage form of claim 1, wherein the pharmaceutically acceptable coating additives are selected from the group consisting of plasticizers, opacifiers, anti-tacking agents, coloring agents, and combinations thereof.

9. The pulsatile-release dosage form of claim 1, wherein the core further comprises one or more rate-controlling polymers.

10. The pulsatile-release dosage form of claim 9, wherein the rate-controlling polymer is selected from the group consisting of cellulose derivatives, polysaccharides, acrylic acid polymers, polyethylene oxides and co-polymers thereof, polypropylene and co-polymers thereof, and combinations thereof.

11. The pulsatile-release dosage form of claim 1, wherein the core further comprises one or more gastro-retentive polymers.

12. The pulsatile-release dosage form of claim 11, wherein the gastro-retentive polymer is selected from the group consisting of polyvinyl alcohol-polyethylene glycol graft copolymers, acrylic acid polymers, and combinations thereof.

13. The pulsatile-release dosage form of claim 1, wherein the core is selected from the group consisting of tablets, capsules, granules, caplets, spheroids, and pellets.

14. The pulsatile-release dosage form of claim 13, wherein the core is a tablet.

15. The pulsatile-release dosage form of claim 13, wherein the core is a capsule.

16. A process for the preparation of the pulsatile-release dosage form of claim 14, wherein the process comprises the steps of:
(a) blending/granulating a drug and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent;
(d) applying the coating composition of step (c) over the tablet core of step (b) to form the pulsatile-release dosage forms.

17. A process for the preparation of the pulsatile-release dosage form of claim 14, wherein the process comprises the steps of:
(a) blending/granulating a drug, one or more rate-controlling polymers, and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent;
(d) applying the coating composition of step (c) over the tablet core of step (b) to form the pulsatile-release dosage forms.

18. A process for the preparation of the pulsatile-release dosage form of claim 14, wherein the process comprises the steps of:
(a) blending/granulating a drug, one or more gastro-retentive polymers, and one or more pharmaceutically acceptable excipients;
(b) compressing the blends/granules of step (a) into a tablet core using appropriate tooling;
(c) dissolving/dispersing a water-soluble polymer, a water-insoluble polymer, a small molecular weight water-insoluble excipient, and one or more pharmaceutically acceptable coating additives in a suitable solvent;
(d) applying the coating composition of step (c) over the tablet core of step (b) to form the pulsatile-release dosage forms.

19. A process for the preparation of the pulsatile-release dosage form of claim 15, wherein the process comprises the steps of:
(a) blending/granulating a drug and one or more pharmaceutically acceptable excipients;
(b) filling the blends/granules of step (a) into a suitable sized capsule to form a capsule core;
(c) dissolving/dispersing a water-soluble polymer, a water-insoluble polymer, a small molecular weight water-insoluble excipient, and one or more pharmaceutically acceptable coating additives in a suitable solvent;
(d) applying the coating composition of step (c) over the capsule core of step (b) to form the pulsatile-release dosage forms.

20. A process for the preparation of the pulsatile-release dosage form of claim 15, wherein the process comprises the steps of:
(a) blending/granulating a drug, one or more rate-controlling polymers, and one or more pharmaceutically acceptable excipients;
(b) filling the blends/granules of step (a) into a suitable sized capsule to form a capsule core;
(c) dissolving/dispersing a water-soluble polymer, a water-insoluble polymer, a small molecular weight water-insoluble excipient, and one or more pharmaceutically acceptable coating additives in a suitable solvent;
(d) applying the coating composition of step (c) over the capsule core of step (b) to form the pulsatile-release dosage forms.

21. A process for the preparation of the pulsatile-release dosage form of claim 15, wherein the process comprises the steps of:
(a) blending/granulating a drug, one or more gastro-retentive polymers, and one or more pharmaceutically acceptable excipients;
(b) filling the blends/granules of step (a) into a suitable sized capsule to form a capsule core;
(c) dissolving or dispersing a water-soluble polymer, a water-insoluble polymer, a small molecular weight water-insoluble excipient, and one or more pharmaceutically acceptable coating additives in a suitable solvent;
(d) applying the coating composition of step (c) over the capsule core of step (b) to form the pulsatile-release dosage forms.
